(19) United States
(12) Patent Application Publication
(10) Pub. No.: US 2022/0180991 A1
(43) Pub. Date: Jun. 9, 2022

(54) SYSTEMS AND METHODS FOR CREATING PERSONALIZED MEDICINES FOR TREATING HEART FAILURE AND HEART DISEASE

(71) Applicant: Acorai AB, Domsten (SE)

(72) Inventor: Filip Ludwig Peters, Domsten (SE)

(73) Assignee: Acorai AB, Domsten (SE)

(21) Appl. No.: 17/113,071

(22) Filed: Dec. 6, 2020

(51) Int. Cl. G16H 20/10; G16H 10/60; G06N 3/08

(52) U.S. Cl. CPC G16H 20/10; G06N 3/08; G16H 10/60

(57) ABSTRACT

Systems and methods for creating personalized medicines for treating heart failure and heart disease that can capture non-invasive intracardiac pressure signals of the user through a micro-electro-mechanical sensor configured to a computing device. Bio-sample user data can also be captured through a bio-sample module. Other components can capture demographic data pertaining to the user. The user's Patient Health Record (PHR) data is captured through a PHR module. The system uses the processed data to create one or more personalized medicines for heart failure and heart disease through a machine learning module which can then be presented on a computing device user interface.

### CROSS-REFERENCES TO RELATED PATENT APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISK APPENDIX

Not Applicable.

### BACKGROUND

### Technical Field

The inventive subject matter presented herein is generally directed towards generative medicine for heart failure and heart disease. More particularly, but not limited to, it is directed to methods including computer-implemented methods for creating personalized medicines for treating heart failure and heart disease guided by intracardiac pressure monitoring.

### Description of the Related Art

Heart disease ending in heart failure is a chronic, progressive, and incurable disease which affects over 30 million people worldwide. Heart failure (HF) and heart disease (HD) are highly prevalent and affect about 1% of persons in their 50's, an amount that progressively rises with age to afflict 10% of persons in their 80's. HD and HF have a complex clinical syndrome of multiple symptoms, functional impairments, and poor health-related quality of life (HRQoL). Progress in the space has led to HF and HD patients living longer but with potentially higher symptom burdens comparable to patients with other chronic diseases such as cancer. Poor symptom controls and poor HRQoL relating to HF and HD are significant drivers of hospitalizations, re-hospitalizations, and death. Typically, intracardiac pressure monitoring is an important aspect of heart failure and heart disease management and treatment. For example, a rise of the intracardiac pressure, such as in the left atrium, is an important early indication of disease progression and an early opportunity for therapeutic intervention. Congestion is a common complication of both heart failure and heart diseases. Typically, patients suffering from pulmonary hypertension can be monitored using intracardiac pressure measurements. Often, HF and HD have precipitating factors and occur in accordance with one another. It is therefore prudent to monitor and treat both HF and HD in conjunction with one another. For example, common precipitating factors for heart failure hospitalizations are pneumonia/respiratory process, myocardial ischemia, arrhythmia, uncontrolled hypertension and non-adherence to diets and medications.

The existing systems and methods for measuring intracardiac pressure are normally used in a hospital or a clinic setting and provide the measured intracardiac pressure when the patient is already in a very critical situation. Existing systems and methods for treating heart failure and heart disease normally require costly hospitalization. A system and method to remotely monitor heart patients and provide them personalized medicines would greatly reduce the number of hospitalizations and extend the lives of the patients. Personalized medicines for heart failure and heart diseases are also financially feasible, as they may reduce the costs of drug development by shortening the drug development cycle. Various systems and methods have been used for heart pressure monitoring. For example, U.S. Pat. No. 8,855,757 issued to Kapoor discloses a system for acquiring the electrical footprint of the heart using electrocardiograms (EKG or ECG) as well as providing for heart rate variability monitoring which is incorporated into a mobile device accessory. The ECG signal is acquired and transmitted to a server via the mobile device offering accurate heart rate variability biofeedback measurement. However, this fails to measure intracardiac pressure and capture the medical history of the patients. It also does not facilitate the patients with any personalized medicines so that the patients can avoid hospital visits. The need for fewer hospital visits is important to reduce the spread of diseases in hospitals amongst patients, which has become especially prescient in modern pandemic scenarios such as COVID-19.

It is therefore desirable to provide systems and methods that can remotely monitor the heart failure and heart disease of the patient in real-time in the absence of medical practitioners. There is furthermore a need for a portable system and method that can effectively reduce patient visits to hospitals by aiding in the creation of personalized medicines.

Thus, in view of the above, there is a long-felt need in the healthcare industry to address the aforementioned deficiencies and inadequacies.

### SUMMARY

Systems and methods for creating one or more personalized medicines for treating heart failure and heart disease are provided and described in connection with at least one of the figures.

An aspect of the present disclosure relates to a system for creating one or more personalized medicines for treating heart failure and heart disease. The system includes a processor and a memory. The memory is communicatively coupled to the processor, wherein the memory stores instructions executed by the processor. The processor with memory is configured to work with a computing device to capture non-invasive intracardiac pressure signals of the user through at least one or more of the following: micro-electro-mechanical sensors, inertial measurement unit sensors, microphonic sensors, electrodes and photoplethysmography sensors. The processor with memory is configured to capture the bio-sample data of the user through a bio-sample module. The processor with memory is configured to capture demographic data pertaining to the user through a demography module. The processor with memory is configured to capture Patient Health Record data of the user through a PHR module. The processor with memory is configured to process the patient data for creating the one or more personalized medicines subject to toxicity levels for heart failure and heart disease through a machine learning module. The patient data includes one or more of the following: non-invasive intracardiac pressure signals, bio-sample data, demographic data, and PHR data. The processor with memory is configured to present the one or more personalized medicines for heart failure and heart disease on a user interface configured with the computing device.

In many embodiments, the intracardiac pressure signals are indicative of pressures within the heart of the user.

In an embodiment, the micro-electro-mechanical sensor computes a change in non-invasive intracardiac pressure as a difference between two or more intracardiac pressure measurements or signal recordings.

In an embodiment, the bio-sample module is configured to help the user collect a bio-sample from salivary glands in a form of saliva.

In an embodiment, the bio-sample module is configured to facilitate the user to place the bio-sample on a reactant paper having one or more reactant properties pertaining to chemical information of the user's body.

In some embodiments, the bio-sample module is configured to aid the user in capturing images of the bio-sample on the reactant paper so that the bio-sample data can be obtained.

In some embodiments, the personal health record (PHR) data comprises a plurality of medical events with a plurality of time stamps.

In many embodiments, the processor is configured to register the user over a communication application by receiving one or more credentials from the user and processing the credentials through a registration module so that access can be provided to the user.

In an embodiment, the processor is configured to estimate toxicity of the one or more personalized medicines through a toxicity estimation module.

In an embodiment, the processor is configured to process data using a generative adversarial network through a machine learning module.

In an embodiment, the memory is configured to capture medicine adherence data of the user through a medicine plan adherence module.

In an embodiment, the processor is configured to communicate between the user and an expert, through a communication module.

In an embodiment, the processor is configured to provide non-pharmacologic treatment recommendations, through a non-pharmacologic treatment module.

In an embodiment, the communication application is executable on the computing device of the user.

In an embodiment, the computing device generates personalized medicines or medical recipes based on a new dataset processed by the machine learning module.

An aspect of the present disclosure relates to a computer-implemented method for creating one or more personalized medicines for treating heart failure and heart disease. The computer-implemented method includes a step of capturing non-invasive intracardiac pressure signals of the user through a micro-electro-mechanical sensor configured to a computing device. The computer-implemented method includes a step of capturing bio-sample data of the user through a bio-sample module. The computer-implemented method includes a step of capturing demographic data pertaining to the user through a demography module. The computer-implemented method includes a step of capturing PHR data of the user through a PHR module. The computer-implemented method includes a step of processing the patient data for creating the one or more personalized medicines for heart failure and heart disease through a machine learning module. The patient data includes non-invasive intracardiac pressure signals, bio-sample data, demographic data, and PHR data. The computer-implemented method includes a step of presenting the one or more personalized medicines for heart failure and heart disease on a user interface configured with the computing device.

In an embodiment, the intracardiac pressure signals are indicative of a pressure within the heart of the user.

In an embodiment, the micro-electro-mechanical sensor computes a change in non-invasive intracardiac pressure as the difference between two or more intracardiac pressure signal recordings.

In an embodiment, the computer-implemented method includes a step of processing PHR data of the user using a generative adversarial network.

In an embodiment, the computer-implemented method includes a step of capturing medicine adherence data of the user through a medicine plan adherence module.

In an embodiment, the computer-implemented method includes a step of communicating between the user and an expert, through a communication module.

In an embodiment, the computer-implemented method includes a step of presenting non-pharmacologic treatment recommendations, through a non-pharmacologic treatment module.

In an embodiment, the bio-sample module is configured to facilitate the user to collect a bio-sample from salivary glands in a form of saliva.

In an embodiment, the bio-sample module is configured to facilitate the user to place the bio-sample on a reactant paper having one or more reactant properties pertaining to a user's body chemical information.

In an embodiment, the bio-sample module is configured to facilitate the user to capture an image of the reactant paper with a bio-sample in order to obtain bio-sample data.

In an embodiment, the personal health record (PHR) data comprising a plurality of medical events having a plurality of time stamps.

In an embodiment, the computer-implemented method includes a step of registering the user over a communication application through a registration module by receiving one or more credentials from the user so that the embodiment can provide access to the communication application.

In some embodiments, the computer-implemented method includes a step of estimating toxicity of the one or more personalized medicines through a toxicity estimation module.

In many embodiments, the communication application is executable on the computing device of the user.

In many embodiments, the computing device generates personalized medicines or medical recipes based on a dataset processed by the machine learning module.

In some embodiments, the personalized medicines or medical recipes are provided directly to the patient.

In many embodiments, the patient is provided with several types of medicines and subsequently provided with instructions on administering the medicines with dosage based on heart failure and heart disease progression data.

In an embodiment, the patient is provided with instructions pertaining to non-pharmacological lifestyle changes based on heart failure and heart disease progression data. Examples of lifestyle changes may relate to nutrition, nutraceutical supplements, antioxidants, weight loss, exercise, meditation, and sleep. Such a non-pharmacologic treatment module may further be configured to trigger treatments and/or preventive measures based on information related to the user's physical activity, e.g. information derived through the accelerometer of the user's phone and/or information from wearable devices such as a digital watch or bracelet.

In an embodiment, the patient is prompted to submit data pertaining to their adherence to the prescribed medicine regimen.

One advantage of the inventive subject matter is that it provides computer-implemented methods and systems for assessing intracardiac pressure and creating personalized medicines for heart failure and heart disease. In an aspect, personalized medicines are generative medicines having the potential of being more accurate, efficient, and with fewer side-effects.

Another advantage of the inventive subject matter is that embodiments provide computer-implemented methods and systems that incorporate reminders, nudges, and notifications into the communication application to help the patients to follow their personalized medicines which may enable the patients to be more inclined to adhere to their medication schedule and increase the efficacy of the medicine.

Yet another advantage of the inventive subject matter is that embodiments provide a computer-implemented method and system that can continuously track the patient's general wellbeing as well as heart failure and heart disease progression.

Another advantage of the inventive subject matter is that embodiments utilize a data-driven approach to provide robustness across patient cohorts in terms of objectivity and interpretability and embodiments may furthermore not be prone to environmental circumstances of clinical settings which may interfere with the communicative process between a clinician and a patient.

Another advantage of the inventive subject matter is that embodiments may incorporate safe natural health medicines into a personalized heart failure and heart disease treatment plan to provide safe medicine to the patients.

Yet another advantage of the inventive subject matter is that embodiments employ machine-learning enabled image analysis to analyze one or more bio-samples. This can be beneficial for many reasons including the fact that any particular bio-sample does not need to be transported long distances to a lab for later analysis which may increase the integrity of the bio-sample as there would be less opportunity for any sample to be interfered with and/or interact with different environments.

Another advantage of the inventive subject matter is that it reduces the time to predict potential symptom reducing medicines to reduce the prevalence of adverse events resulting from heart failure and heart disease.

Another advantage of the inventive subject matter is that embodiments create personalized medicines for heart failure and heart disease using generative machine learning methods and non-invasive cardiac pressure monitoring in adaptive clinical trial settings.

Another advantage of the inventive subject matter is that the severity of data leakage risks may be reduced.

Other advantages will become apparent to those skilled in the art upon viewing the drawings and reading the detailed description without departing from the spirit and the scope of the claimed subject matter. The drawings and detailed description presented herein are to be regarded as illustrative in nature and not in any way as restrictive.

### BRIEF DESCRIPTION OF THE DRAWINGS

In the figures, similar components and/or features may have the same reference labels. Further, various components of the same type may be distinguished by following the reference label with a second label that distinguishes among the similar components. If only the first reference label is used in the specification, the description applies to any one of the similar components having the same first reference label irrespective of the second reference label.

FIG. 1 illustrates a block diagram of the present system for creating one or more personalized medicines for treating heart failure and heart disease in accordance with embodiments of the claimed subject matter;

FIG. 2 illustrates a block diagram of the various modules within a memory of a computing device in accordance with embodiments of the claimed subject matter;

FIG. 3 illustrates a perspective view of receiving a bio-sample from a user in accordance with embodiments of the claimed subject matter;

FIG. 4 illustrates a flow diagram of machine learning training and feature generation through a division of columns by one another in accordance with embodiments of the claimed subject matter;

FIG. 5 illustrates a flow diagram of generating personalized medicines by employing generative adversarial networks and/or other non-linear solving methods in accordance with embodiments of the claimed subject matter;

FIG. 6 illustrates a flowchart of a computer-implemented method for creating one or more personalized medicines for treating heart failure and heart disease, in accordance with embodiments of the claimed subject matter; and

FIG. 7 illustrates a rear view of an exemplary smartphone with the micro-electro-mechanical sensor provided on the top rear surface of the smartphone in accordance with embodiments of the claimed subject matter.

### DETAILED DESCRIPTION

The present description is best understood with reference to the detailed figures and description set forth herein. Various embodiments of the present systems and methods have been discussed with reference to the figures. However, those skilled in the art will readily appreciate that the detailed description provided herein with respect to the figures is provided for illustrative purposes as the present systems and methods may extend beyond the described embodiments. For instance, the teachings presented and the needs of a particular application may yield multiple alternative and suitable approaches to implement the functionality of any detail of the systems and methods described herein. Therefore, any approach to implement the present system and method may extend beyond certain implementation choices described in the embodiments.

According to embodiments herein, the methods may be implemented by performing or completing manually, automatically, and/or a combination of thereof. The term “method” refers to manners, means, techniques, and procedures for accomplishing any task including, but not limited to, those manners, means, techniques, and procedures either known to the person skilled in the art or readily developed from existing manners, means, techniques and procedures by practitioners of the art to which the inventive subject matter belongs. Persons skilled in the art will envision many other possible variations within the scope of the systems and methods described herein.

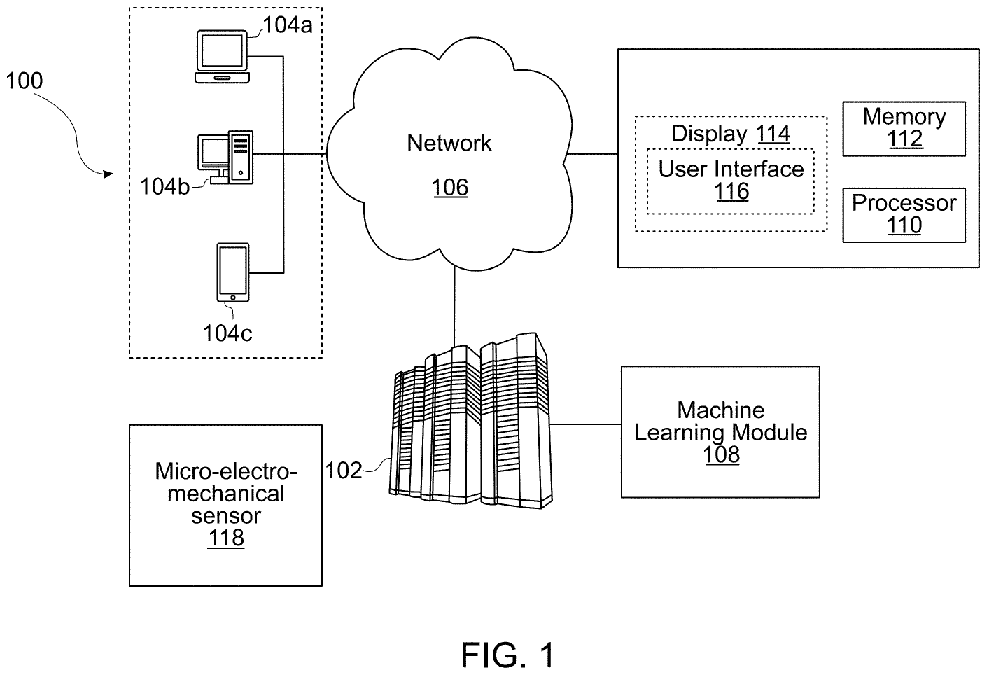

FIG. 1 illustrates a block diagram of the present system 100 for creating one or more personalized medicines for treating heart failure and heart disease in accordance with embodiments of the claimed subject matter. The system 100 includes a processor 110, a memory 112, and a server 102. The memory 112 is communicatively coupled to the processor 110, wherein the memory 112 stores instructions executed by the processor 110. The memory 112 may be a non-volatile memory or a volatile memory. Examples of non-volatile memory may include, but are not limited to flash memory, a Read Only Memory (ROM), a Programmable ROM (PROM), Erasable PROM (EPROM), and Electrically EPROM (EEPROM) memory. Examples of volatile memory may include but are not limited Dynamic Random-Access Memory (DRAM), and Static Random-Access memory (SRAM). The processor 110 may include at least one data processor for executing program components for executing user- or system-generated requests. Processor 110 may include specialized processing units such as integrated system (bus) controllers, memory management control units, floating point units, graphics processing units, digital signal processing units, etc. Processor 110 may include a microprocessor, such as AMD® ATHLON® microprocessor, DURON® microprocessor OR OPTERON® microprocessor, ARM's application, embedded or secure processors, IBM® POWERPC®, INTEL'S CORE® processor, ITANIUM® processor, XEON® processor, CELERON® processor or other line of processors, etc. Processor 110 may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), and the like.

Processor 110 may be disposed of in communication with one or more input/output (I/O) devices via an I/O interface. An I/O interface may employ communication protocols/methods such as, without limitation, audio, analog, digital, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.n/b/g/n/x, Bluetooth, cellular (e.g., code-division multiple access (CDMA), high-speed packet access (HSPA+), global system for mobile communications (GSM), long-term evolution (LTE), WiMax, or the like), or any other suitable protocol known to those skilled in the art.

In many embodiments, the system 100 requires a user to register on a communication application configured within one or more computing devices 104 (for example, a laptop 104a, a desktop 104b, and a smartphone 104c.) In other embodiments, the system 100 does not require the registration of the user, for example for use in an emergency situation in which a patient is being monitored during transport to a medical facility for treatment or in a triage screening situation. Other examples of the computing devices 104, may include but are not limited to a phablet and a tablet. A user may include a patient such as a patient using the communication application and one or more computing devices 104, a medical professional, a third party, and the computing device itself. The processor 110, memory 112, server 102, and the computing devices 104 are communicatively coupled over a network 106. A network 106 may be a wired or a wireless network with examples including but not limited to the internet, a Wireless Local Area Network (WLAN), a Wi-Fi network, a Long Term Evolution (LTE) network, a Worldwide Interoperability for Microwave Access (WiMAX) network, and a General Packet Radio Service (GPRS) network.

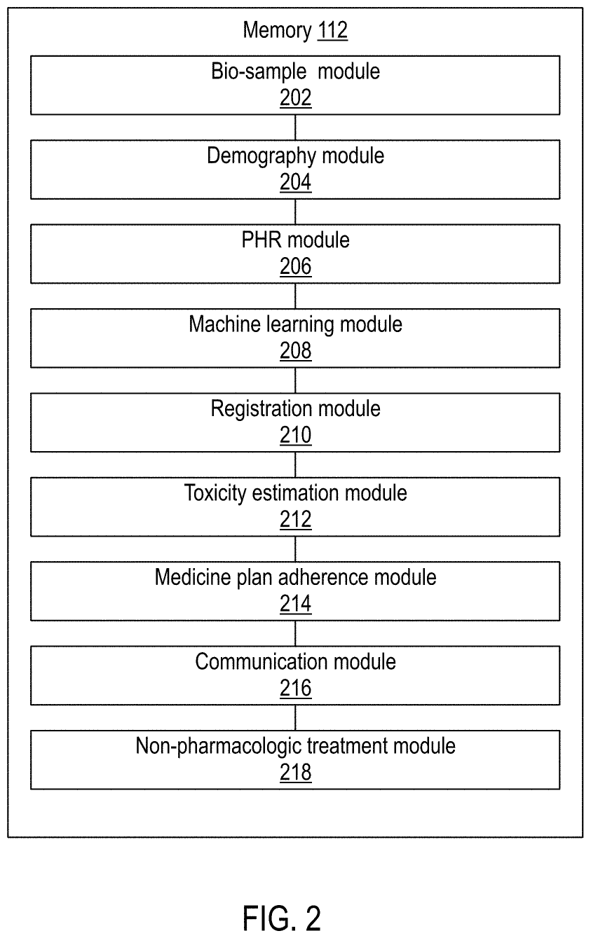

In many embodiments, memory 112 includes modules that enable the system 100 to generate one or more personalized medicines and/or treatment regimens for heart failure and heart disease. Some of these modules are illustrated in FIG. 2. The system 100 may also include a display 114 having a User Interface (UI) 116 for use, for example by the user or an administrator, to initiate a request to view the personalized medicines and provide various inputs to the system 100. In an embodiment, the User Interface (UI or GUI) 116 is a convenient interface for accessing the information related to various personalized medicines, including the patient data, and an interface for wellbeing questionnaires for the user. Display 114 can also be used to display personalized medicines to the users. In some embodiments, the functionality of the system 100 may be configured to run in more than computing devices 104.

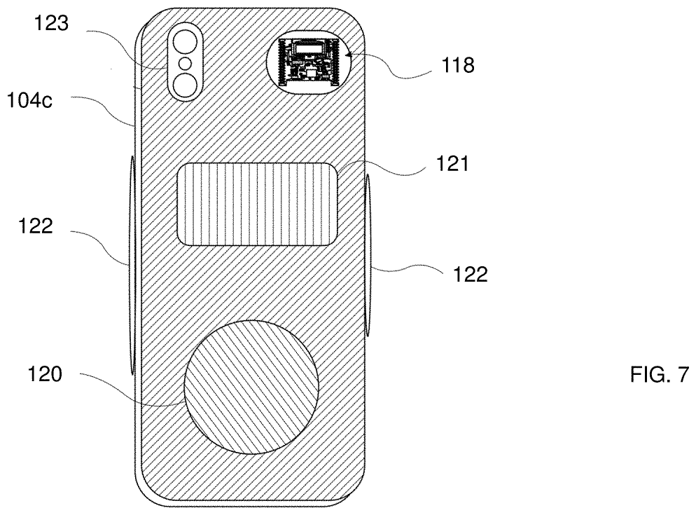

FIG. 2 illustrates a block diagram of the various modules using memory 112 of a computing device 104 in accordance with several embodiments. FIG. 2 is explained in conjunction with FIG. 1. Embodiments include the use of a bio-sample module 202, a demography module 204, a personal health record (PHR) module 206, a machine learning module 208, a registration module 210, a toxicity estimation module 212, a medicine plan adherence module 214, a communication module 216, and a non-pharmacologic treatment module 218. These modules are software components which include programs that contain one or more routines or algorithms that enable the processing of inputs and outputs. In other embodiments, hardware and/or hardware and software configurations may be used to accomplish the same or similar results. Embodiments can be used to capture non-invasive intracardiac pressure signals of the user through a micro-electro-mechanical sensor 118 in conjunction with the computing device 104. According to several embodiments, the non-invasive intracardiac pressure signals can be captured by one or more of the following: the microphonic sensor 120, the Inertial Measurement Unit (IMU) sensor 121, the electrodes 122 or the photoplethysmography sensors 123. In an embodiment, the intracardiac pressure signals are indicative of a pressure within the heart of the user. In an embodiment, the micro-electro-mechanical sensor 118 computes a change in non-invasive intracardiac pressure measurement as a difference between two or more intracardiac pressure measurements or signal recordings. In an embodiment, the micro-electro-mechanical sensor 118 is a pressure sensor. In an embodiment, the micro-electro-mechanical sensor 118 can be placed and secured on the top rear surface of the computing device 104 as illustrated in FIG. 7. Further, the micro-electro-mechanical sensor 118 is electrically coupled with the computing device 104 to transmit the captured non-invasive intracardiac pressure signals. In some embodiments, one or more micro-electro-mechanical sensors for pressure monitoring may be implanted into the pulmonary artery of a patient to monitor and transmit data. In another embodiment, a micro-computer may be implanted into the left atrium of a patient to monitor and transmit pressure data and in other embodiments the micro-computer may also store and process pressure data and any other related information.

Embodiments can be configured to capture demographic data pertaining to the user through the demography module 204. In an embodiment, the user is prompted to enter his/her demographic data. In use, demographic data such as gender, ethnicity, weight, and height may be important in helping determine the efficacy of any personalized medicine and/or any related treatment regimens.

Many embodiments can be configured to capture PHR data of the user through the PHR module 206. In some of these embodiments, a patient user is prompted to enter his/her patient data. Patient data such as gender, ethnicity, weight, height, and previous medical history may be important in determining the efficacy of personalized medicine and/or treatment regimens. Patient health records may be analyzed using Natural Language Processing machine learning methods to identify variables that may be important in determining the patient's receptibility to certain medicines, previous treatment bias and/or mistakes, and other information that may not be readily obvious from a non-data machine learning approach.

Several embodiments may be used to process the patient data to create one or more personalized medicines utilizing one or more toxicity levels in relation to heart failure and heart disease through the machine learning module 208. The patient data includes but is not restricted to one or more of the following: intracardiac pressure signals, bio-sample data, demographic data, and PHR data. In many embodiments, the PHR data includes a plurality of medical events having a plurality of time stamps. Embodiments can be configured to present the one or more personalized medicines for heart failure and heart disease on the user interface 116 in conjunction with the computing device 104. PHR data may further comprise non-pharmacological information pertaining to nutrition, nutraceutical supplements, antioxidants, weight loss, exercise, meditation, and sleep. PHR data may also include pharmacological history, other treatment history, co-morbidities, and other health conditions of the patient. Additionally, PHR data may include genetic information related to the patient.

In many of the embodiments, a user can register over the communication application through the registration module 210 with one or more provided credentials allowing the user to access communication application. Examples of the credentials include but are not limited to: a user name, password, age, gender, phone number, email address, and location of the user. In several embodiments, the toxicity of the one or more personalized medicines can be estimated and communicated through a toxicity estimation module. In another embodiment, the measured toxicity may also include measurements of other adverse conditions with corresponding effects both in a continuous or binary data format. In many embodiments, the communication application is executable on the computing device of the user and implemented on one or more operating systems such as Android®, iOS®, Windows®, etc. Also in many embodiments, the communication application is commercialized as an application for creating personalized medicines for heart failure and heart disease. The embodiments may consist of a software application, a mobile application, a web application, a hardware solution, a hardware/software solution or any other suitable application or solution. In some embodiments, the computing device 104 generates personalized medicines, recipes or regimens based on one or more datasets processed by one or more machine learning modules. In some embodiments, the processor 110 is configured to process data using a generative adversarial network through a machine learning module 108. In some embodiments, a user's medicine adherence data is captured with a medicine plan adherence module 214. In many embodiments, the processor 110 is configured to communicate between the user and an expert via a communication module 210. In an embodiment, the processor 110 is configured to provide non-pharmacologic treatment recommendations through a non-pharmacologic treatment module 218.

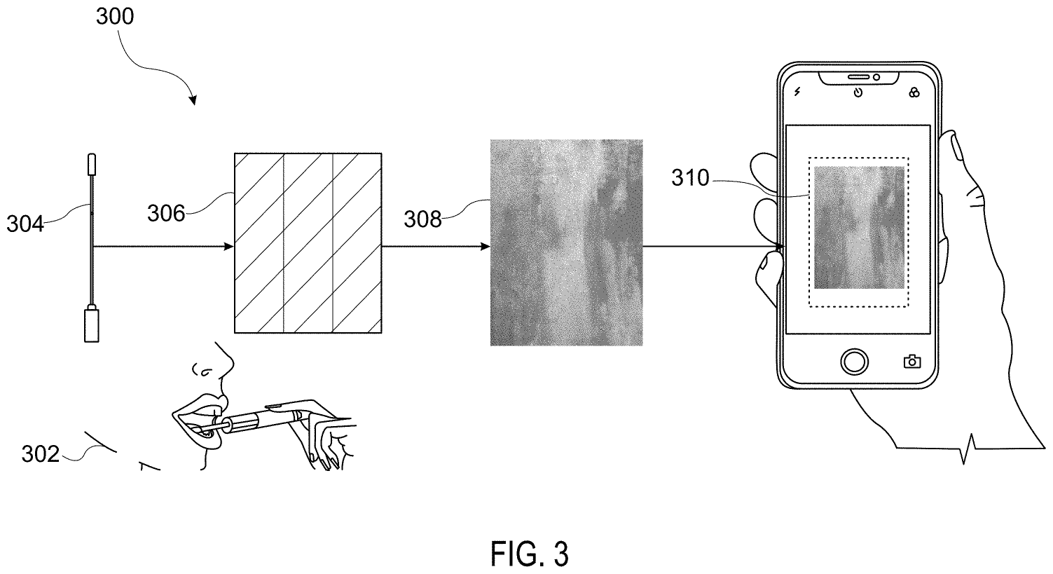

FIG. 3 illustrates exemplary steps involved in receiving a bio-sample from the user 302 in accordance with at least one embodiment. FIG. 3 is explained in conjunction with FIG. 2. The bio-sample module 202 is configured to capture the bio-sample data of the user utilizing the components of the embodiments. As shown, a tool 304, such as a swab, is used to collect saliva from the user's salivary glands and subsequently used by the embodiment as a bio-sample for processing by the bio-sample module 202.

In several embodiments, the user places the bio-sample (e.g. saliva or other biological sample) on a portion of reactant paper 306. The reactant paper 306 has one or more reactant properties pertaining to chemical information of the user's body and in many of these embodiments it changes color 308 upon receiving the bio-sample. In these embodiments, the reactant paper 306 is used with the bio-sample module 202 so that an image 310 of the reactant paper 306 is captured by the embodiment when the user places the bio-sample on the reactant paper 306 within the bio-sample module 202. In other embodiments, the reactant paper 306 can be positioned externally to the bio-sample module 202 and imaged.

In many of the embodiments, the patient may be prompted to record bio-sample data at the same time as the intracardiac pressure is being measured so that data regarding relationships between the microbiome and intracardiac pressure can be obtained. It is well known that genetic factors play a role in heart failure and heart disease. Such information may be used in conjunction with the described bio-sample collection methods.

It is also known that changes in the composition of gut microbiota, referred to as dysbiosis, might play a pivotal role in the development of HF. HF-associated decreased cardiac output resulting in bowel wall oedema and intestine ischemia can alter the gut structure, permeability, and function. Such changes may favor bacterial translocation, exacerbating HF and HD pathogenesis in several ways including but not limited to activation of systemic inflammation. In many of the embodiments, bio-samples can be used to identify one or more microbiomes. The information derived from the processing of the bio-samples can be used to aid in the evaluation of heart failure and heart disease, for instance deriving correlations in the information with other data.

Many embodiments are used to identify various elements and characteristics pertaining to heart failure and heart disease in a contained environment leading to less risk of data leakage and contamination.

According to several embodiments, the user is prompted to collect saliva from the user's salivary glands so it that saliva can be placed on reactant paper 306 for further use as a bio-sample. Different parts of the reactant paper 306 may have different reactant properties pertaining to different chemical information from the body. Said reactant material may include one or more of the following: ketostix strips, API 20E test strips, nitrocellulose-based membranes, glass cellulose-based absorbent pads, pH strips, and any other suitable reactant medium. In some embodiments, fluorescent or another suitable type of light may be used to extract data pertaining to different reactant properties of the bio-sample.

In other embodiments, the bio-sample is placed into a soluble substance that can visually interact with the bio-sample. Upon successful completion of a bio-sample collection and interaction process, the user is prompted to take an image of the reactive process. In some embodiments, the image may be taken without any prompt or user action so that the image is automatically captured. The bio-sample image acquisition process may be done within an application from a handheld electronic device, a computer device equipped with a camera, or any other suitable photographic or video device.

Recent research has shown that saliva microbiome profiles are minimally affected by collection methods or DNA extraction protocols. These protocols may reveal a vast amount of information from a human body. In these embodiments, the bio-sample image data is uploaded to a server and combined with other data to provide a final dataset. Other embodiments may only use the bio-sample image data for processing and creation of results.

In an embodiment, the user is presented with one or more questions pertaining to the type of HF and HD with one or more selectable answers that may aid in the risk assessment of HF and HD. For example, in one embodiment the patient selects at least one answer from the plurality of corresponding selectable answers to the one or more questions to obtain and assess the heart assessment data. Exemplary questions may comprise non-pharmacological information pertaining to nutrition, nutraceutical supplements, antioxidants, weight loss, exercise, meditation, and sleep. Other exemplary questions may include general physical and mental wellbeing questions as it pertains to the patient.

According to several embodiments, a server 102 is communicatively coupled to the memory 112 and other components over a network 106. The server 102 may process the final dataset by applying a machine learning module 108. The server 102 is configured to generate personalized medicines or tailored medical recipes and/or regimens based on one or more datasets processed by the machine learning module 108. In some of these embodiments, the server 102 is configured to create personalized medicines or generative medicines based on the final dataset processed by the machine learning module. In many of these embodiments, the server 102 is configured to transmit the personalized medicines to the one or more computing devices 104 over the network 106. In an embodiment, the final dataset is uploaded to the server 102 and analyzed by various machine learning algorithms associated with the machine learning module 108 to generate one or more tailored diets based on the information associated with the user. The tailored diets may then be produced as medical products or other food products that can also be shipped directly to the users. In this way, the embodiments can provide both a health assessment mechanism and one or more tailored diets for the user.

In several embodiments, server 102 estimates the toxicity of one or more generative medicines with the estimations transmitted to the computing device through a toxicity estimation module. In some of these embodiments, the toxicity may be estimated based on feedback data received from one or more patients after consumption of the created generative medicines and regimens. A predictive toxicity model may be trained based on the toxicity feedback data and then a second set of generative medicines may be selected subject to toxicity constraints. In another embodiment, previously existing databases of molecular compositions and toxicity may be used to train the predictive toxicity model. In another embodiment, rodent models of toxicity may be used to estimate toxicity in human subjects. In another embodiment, measured toxicity may comprise measuring other adverse effects, in a continuous or binary data format. In other embodiments, patient data pertaining to historical reactions to a variety of medicines including the current medicine being proposed may be incorporated into a toxicity analysis. In some embodiments, the patient history data may be analyzed in one or more ways including but not limited to manually, through Natural Language Processing, and any other suitable machine learning methods in order to determine personalized patient toxicity risks.

In some embodiments having genetic data acquisition capability, genetic data can be used to reveal the inherited or acquired genetic characteristics of the patient, for instance using data obtained from chromosomal, deoxyribonucleic acid (DNA) or ribonucleic acid (RNA) analysis.

According to some embodiments, the machine learning module 108 uses machine learning algorithms that may be trained to prioritize the diversity of medical recipes and regimens to broaden the number of unique data points on which the machine learning algorithms can draw from.

In many embodiments, patient data may be clustered to create fewer categories within the recipe or regimen generation process. Examples of such clustering methods include but are not limited to: Affinity Propagation, Agglomerative Clustering, BIRCH, DBSCAN, K-Means, Mini-Batch, K-Means, Mean Shift, OPTICS, Spectral Clustering, and Gaussian Mixture Models.

Several embodiments include a medicine plan adherence module in which a sensor-equipped medicine container may be provided to the patient. The sensor-equipped medicine container can measure the weight of the medicine to provide indications as to what degree the patient is adhering to the treatment plan and as to the times the patient has been taking their medicine. The sensor-equipped medicine container may also be equipped with an alarm in the form of a sound, light or touch so that it may be activated to remind the patient to take their medicine at certain times throughout the day and night. Additional data pertaining to the patient's adherence to their medical plan, treatments and regimens may be collected, stored and used in future analyses. Examples of data collected may include information derived from patient self-assessment questionnaires, images of the medical container and the prescribed dosage, bio-sample analysis data for assessing the presence of the medicine in the patient's body, and data from any number and type of sensors used with the prescribed medicine, for example to determine amounts and quality of use.

In several embodiments, the bio-sample module is configured to allow a user to submit one or more bio-samples consisting of biological materials including but not limited to: blood, urine, tissue, cells, and cell cultures. In some embodiments, the bio-sample module is configured to allow a user to place a bio-sample in a container having a soluble substance with one or more reactant properties that pertain to biological information of the user's body. In an embodiment, the bio-sample module is configured to allow the user to place the bio-sample in an airtight container for preservation so that it can be later analyzed in a laboratory setting. Other embodiments include any suitable type of container known to those skilled in the art that can be configured to obtain and preserve any type of bio-samples and that can accommodate one or more sensors, if desired.

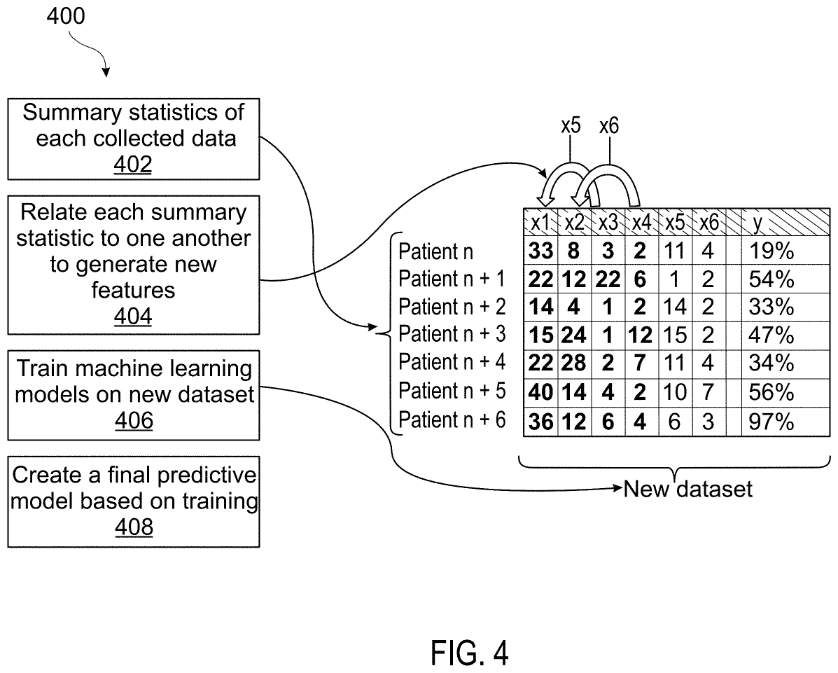

FIG. 4 illustrates a flow diagram 400 of machine learning training and feature generation through a division of columns by one another in accordance with embodiments of the claimed subject matter. Block 402 depicts the summary statistics of each collected data. Block step 404 depicts how each summary statistic is related to one another to generate new features. At block 406, machine learning models are trained on a new dataset. At block 408, a final predictive model is created based on training. The Y-variable in the column depicts an absolute level of heart health and/or change in heart health and/or the efficacy of personalized medicines.

In many embodiments, the machine learning model is trained to predict the effects of certain medicines on heart health progression. In exemplary embodiment, a final dataset is created from data collected from the user acquired in previous steps or stages from the user following directions from previously provided treatments, recipes, and regimens.

In this embodiment, this dataset, which may be construed as the ‘x-variables’, can be combined with data pertaining to the heart health progression of each patient, which data is indicated as the ‘y-variable’ in the machine learning process. The heart health progression data for each patient may be collected one or more days after the patient has been given their medicine.

The machine learning methods include, but are not limited to, decision tree-based machine learning methods, artificial neural networks, convolutional neural networks, logistic regression, naive Bayes, nearest neighbor, support vector machines, boosted tree learning methods, and generative neural networks.

Additionally, embodiments integrate two technology developments which are affecting healthcare in significant ways. The first is the mandate to move the continuum of care outside of the hospital. For example, the internet of things (IoT) allows for massive instrumentation of the healthcare experience regardless of the location of the patient and the health care provider. The second development is the use of machine learning and other deep analytics capabilities to increase the quality of care. As healthcare organizations begin to look into “smart” technologies, the inventive subject matter provides opportunities never before seen in the healthcare industry.

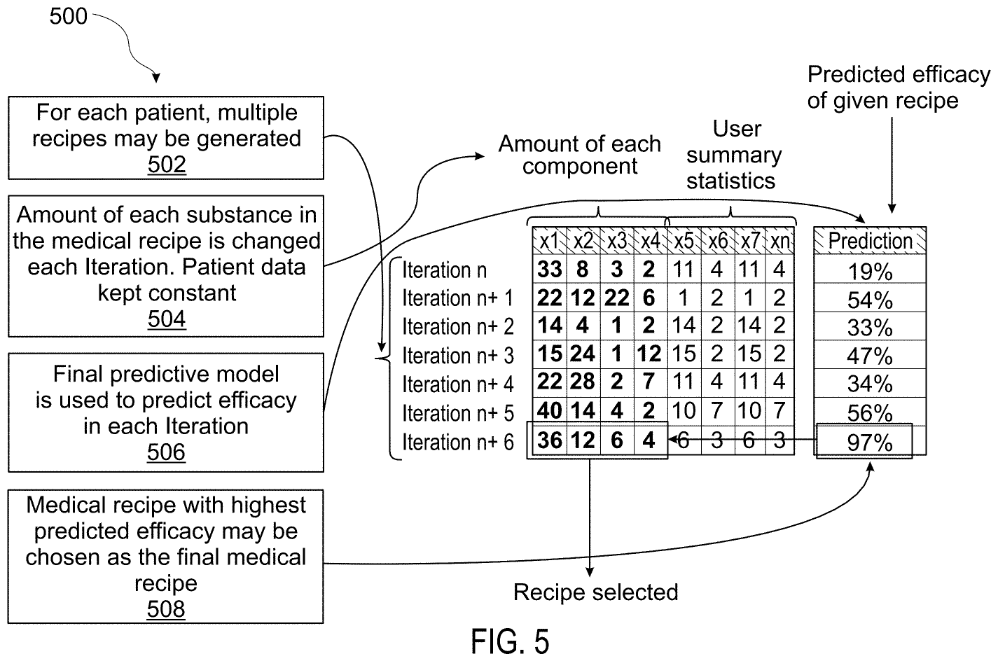

FIG. 5 illustrates a flow diagram 500 of the generation of personalized medicines by employing generative adversarial networks and/or other non-linear solving methods, in accordance with at least one embodiment. Block 502 depicts multiple medical recipes generated for each user. Block 504 depicts the amount of each substance in the medical recipe changed with each iteration and with the patient data being kept constant. At block 506, the final predictive model is used to predict the efficacy of each iteration. At block 508, the medical recipe with the highest predicted efficacy is chosen as the final medical recipe and recommended for the user.

In many embodiments, each of the patient recipes are created and the medical recipe, along with any data transformation functions such as average, median, or sum of several recipes, along with the highest predicted efficacy data, may be selected as the final medical recipe to be recommended for the patient.

In these embodiments, the solving systems include, but are not limited to, one or more of the following: brute force search algorithms, linear solving methods, non-linear solving methods such as genetic algorithm solvers and random number generators. In these embodiments, the data collected from the user is held constant while the values for each possible component in the recipe are custom tailored for each treatment regimen.

Additionally, certain constraints may be imposed on the solving methods. Examples of constraints may include individual ingredient and total medical recipe amounts in terms of weight and the types of compounds that can be safely combined and specific combinations of substances that might be deemed inappropriate. For example, the final recipe needs to be consumable by the patient and potentially toxic combinations of compounds as well as toxic amounts need to be avoided.

Substances used in the medical recipe generation process may include any natural health product, any naturally-occurring substance, or any other suitable substance known to those skilled in the art. Exemplary substances include, but are not limited to, Ginkgo Biloba, Ketogenic Diet, Sulforaphane, Genistein, Ketogenic medium-chain triglyceride drink (MCT drink), Mediterranean Diet, Low-fat Diet, MMFS-205-SR, Omega-3 treatment, MitoQ, EGCG, melatonin, Probiotic supplemented intervention, HYMN Ketone Esther drink, Grape Powder, Mega natural-Az Grapeseed Extract, vitamin E in combination with Selegiline, and any of Vitamins A-K. Substances may also include different doses of certified heart health medicines such as ACE inhibitors, angiotensin-2 receptor blockers (ARBs), beta-blockers, mineralocorticoid receptor antagonists, diuretics, ivabradine, sacubitril valsartan, hydralazine with nitrate and digoxin.

Other potential substances that may be used with embodiments include natural medical substances that seek to relax and open up blood vessels. These compounds can make it easier for the heart to pump blood around the body. Natural medicines that reduce oxidative stress, inflammation, and autoimmune dysfunction may also be included. Embodiments may also include any suitable amount of prescription medicine.

In many embodiments, approaches using generative adversarial neural networks (GANs) may be employed to generate potential regimens and/or recipes based on the data submitted by a user. Such GANs may include Deep Convolutional GANs (DCGANs), Wasserstein GANs (WGANs), Self-Attention GANs (SAGANs), and BigGANs. Variational autoencoders may also be used in the training process. The described approaches may also be useful in overcoming the overfitting of the data.

In many of the embodiments, follow up information from the user may be collected so that further analysis of the metadata associated with the predicted recipes can be used to evaluate the regimens and recipes that were recommended to the user and implemented.

Several embodiments can analyze future variations of the recipes and regimens by performing comparisons of predicted results with actual results. The results of the analysis can be used to make adjustments to future predictions that utilize the machine learning model for the creation of new recipes.

In many embodiments, the placebo effects pertaining to a trial may be analyzed and progression data may be adjusted for these effects so that future recipe and regimen generation efficacy can be improved.

In some embodiments, users can connect via voice or via video conference to a health care professional to discuss treatment options and receive recommendations and psychotherapeutic treatment methods including but not limited to cognitive behavioural therapy, psychoanalysis, and psychosynthesis.

Embodiments can also connect the user to a medical professional such as a cardiologist or other expert within the field of medicine for consultations. In some embodiments, communication between the expert and the user may be performed through an automated robot response system driven by artificial intelligence including examples such as chatbots. The automated response system may be trained to automatically answer commonly asked questions and self-learn new material based on current communications with medical professionals for use in future sessions.

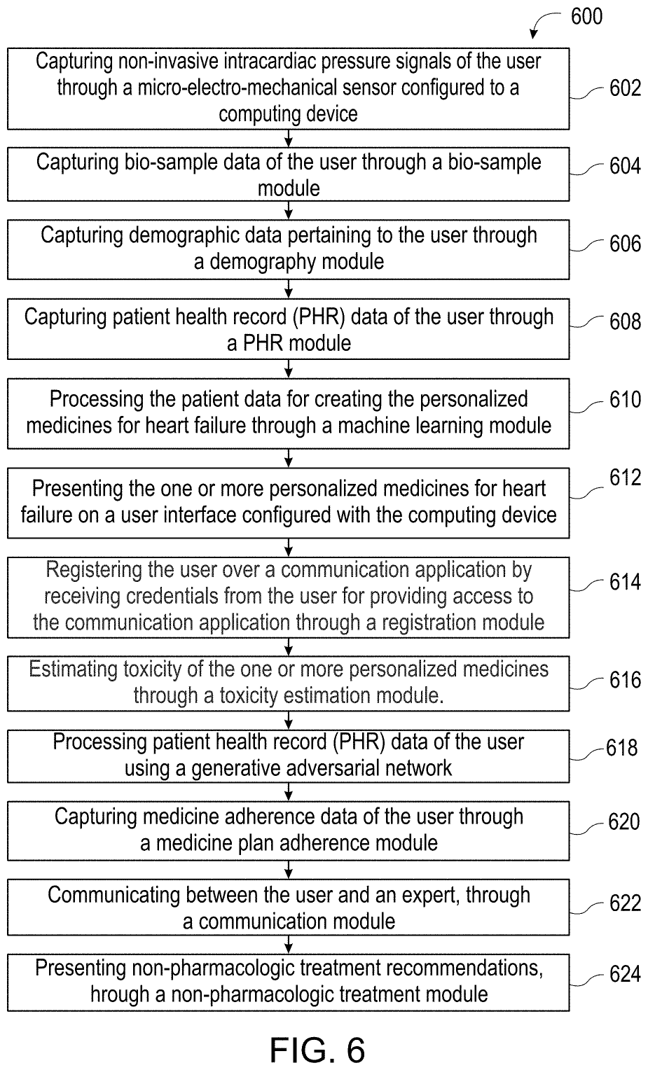

FIG. 6 illustrates a flowchart 600 of exemplary computer-implemented steps used for creating one or more personalized medicines for treating heart failure and heart disease in accordance with embodiments of the claimed subject matter.

These embodiments include a step 602 for capturing a user's non-invasive intracardiac pressure signals using a micro-electro-mechanical sensor in communication with a computing device. In these embodiments, the intracardiac pressure signals are indicative of a pressure within the heart of the user. In some embodiments, the micro-electro-mechanical sensor computes a change in intracardiac pressure as the difference between two or more intracardiac pressure signal recordings.

Also included in these embodiments is step 604 for capturing a user's bio-sample data with a bio-sample module. In these embodiments, the bio-sample module is configured so that a user's saliva from salivary glands can be collected from the user and used as a bio-sample. The user places the bio-sample on a reactant paper having one or more reactant properties pertaining to chemical information of the user's body. The reactant paper is used with the bio-sample module which captures an image of the reactant paper after it is placed within the bio-sample module. In some embodiments, the bio-sample and reactant paper may be positioned and used externally to the bio-sample module.

Step 606 captures demographic data pertaining to the user through a demography module and step 608 captures a user's PHR data with a PHR module. In some embodiments, the PHR data is comprised of one or more medical events with each event having one or more time stamps. Step 610 processes the patient data so that it can be used to create one or more personalized medicines for heart failure and heart disease utilizing a machine learning module. The patient data includes intracardiac pressure signals, bio-sample data, demographic data, and PHR data. Step 612 presents the one or more personalized medicines for heart failure and heart disease on a user interface configured to be used with the computing device.

Next, step 614 registers the user through a registration module over a communication application by receiving one or more credentials from the user so that the use is able to access the communication application. In many of these embodiments, the communication application is executable on the computing device of the user.

Step 616 estimates the toxicity of one or more personalized medicines utilizing a toxicity estimation module. In many of the embodiments, the computing device generates personalized medicines or medical recipes based on one or more new datasets processed by the machine learning module.

Step 618 processes the user's PHR data utilizing a generative adversarial network and step 620 captures the user's medicine adherence data utilizing a medicine plan adherence module. Step 622 allows communication between the user and an expert utilizing a communication module and step 624 presents non-pharmacologic treatment recommendations utilizing a non-pharmacologic treatment module.

The described systems and methods can be utilized to make personalized medicine services for heart failure and heart disease affordable and accurate. Further, embodiments incorporate safe, natural health medicines into personalized heart failure and heart disease treatment plans to provide safer medicine to patients. Embodiments may be applied with other diseases where drug discovery may have failed and where there is little clarity into disease pathology and/or there are few successful known compounds that can be used to train machine learning algorithms In many embodiments where there is an absence of physical clinical sites, a trial may be conducted in a decentralized fashion and medicine may be provided directly to the patients. Many embodiments create personalized medicines for heart failure and heart disease using generative machine learning methods and non-invasive cardiac pressure monitoring in adaptive clinical trial settings.

Unless otherwise defined, all terms (including technical and scientific terms) used in this disclosure have the same meaning as commonly understood by one of ordinary skill in the art to which this disclosure belongs. It is to be understood that the phrases or terms employed in this disclosure is for the purpose of description and not of limitation. As will be appreciated by one of skill in the art, the present disclosure may be embodied as a device, system, method, computer program and/or any combination of the aforementioned.

Further, embodiments may include one or more forms of a computer program product on a computer-readable storage medium having computer-usable program code embodied in the medium. The disclosed systems and methods have been described above with reference to specific examples but other embodiments within the scope of the claimed subject matter are equally possible. The scope of the disclosure may only be limited by the appended patent claims. Even though modifications and changes may be suggested by the persons skilled in the art, it is the intention of the inventors and applicants to embody within the patent warranted heron all the changes and modifications as reasonably and properly come within the scope of the contribution the inventors to the art. The scope of the inventive subject matter is ascertained with the claims as submitted with the specification.

What is claimed is:

1. A computer-implemented method for creating one or more personalized medicines for treating heart failure and heart disease, the computer-implemented method comprising the steps of:
capturing, by the one or more processors, a user's non-invasive intracardiac pressure measurements through a computing device;
capturing, by the one or more processors, demographic data pertaining to the user through a demography module;
processing, by the one or more processors, patient data for creating the one or more personalized medicines for heart failure and heart disease through a machine learning module; and
presenting, by the one or more processors, the one or more personalized medicines for heart failure and heart disease.

2. The computer-implemented method of claim 1 further comprising the step of capturing, by the one or more processors, the user's bio-sample data through a bio-sample module.

3. The computer-implemented method of claim 1 further comprising the step of capturing, by the one or more processors, the user's Patient Health Record (PHR) data through a PHR module.

4. The computer-implemented method of claim 1 further comprising the step of registering the user utilizing a registration module by receiving, processing and storing one or more credentials from the user so that access can be provided to the user.

5. The computer-implemented method of claim 1 further comprising the step of estimating, by the one or more processors, toxicity of the one or more personalized medicines through a toxicity estimation module.

6. The computer-implemented method of claim 1 further comprising the step of processing, by the one or more processors, user patient data using a generative adversarial network.

7. The computer-implemented method of claim 1 further comprising the step of capturing, by the one or more processors, the user's medicine adherence data through a medicine plan adherence module.

8. The computer-implemented method of claim 1 further comprising the step of facilitating communications, by the one or more processors, between the user and an expert through a communication module.

9. The computer-implemented method of claim 1 further comprising the step of presenting, by the one or more processors, non-pharmacologic treatment recommendations, through a non-pharmacologic treatment module.

10. The computer-implemented method of claim 1, wherein the computing device further comprises a micro-electro-mechanical sensor for computing changes in non-invasive intracardiac pressure measurements using the differences between two or more intracardiac pressure measurements.

11. The computer-implemented method of claim 1, wherein the patient data comprises one or more of the following types of data: non-invasive intracardiac pressure measurements, demographic data, bio-sample data, and PHR data.

12. A computer-implemented method for creating one or more personalized medicines for treating heart failure and heart disease, the computer-implemented method comprising:
capturing, by the one or more processors, non-invasive intracardiac pressure signals of the user through a micro-electro-mechanical sensor configured to a computing device;
capturing, by the one or more processors, bio-sample data of the user through a bio-sample module;
capturing, by the one or more processors, demographic data pertaining to the user through a demography module;
capturing, by the one or more processors, PHR data of the user through a PHR module;
processing, by the one or more processors, patient data for creating the one or more personalized medicines for heart failure and heart disease through one or more of the following: a machine learning module, wherein the patient data comprising intracardiac pressure, bio-sample data, demographic data, and PHR data; and
presenting, by the one or more processors, the one or more personalized medicines for heart failure and heart disease on the computing device.

13. The computer-implemented method of claim 12, wherein the bio-sample module is configured so that a user's saliva from salivary glands can be collected from the user and used as a bio-sample.

14. The computer-implemented method of claim 12 wherein the user's bio-sample is placed on a reactant paper having one or more reactant properties pertaining to chemical information of the user's body wherein the information is used as bio-sample data.

15. The computer-implemented method of claim 12 wherein the bio-sample module further captures an image of the reactant paper to obtain the bio-sample data.

16. A system for creating one or more personalized medicines for treating heart failure and heart disease, the system comprising:
a processor;
a memory communicatively coupled to the processor, wherein the memory stores instructions executed by the processor, wherein the system is configured to:
capture non-invasive intracardiac pressure signals of the user through a micro-electro-mechanical sensor configured to a computing device;
capture demographic data pertaining to the user through a demography module;
process patient data for creating the one or more personalized medicines for heart failure and heart disease through a machine learning module; and
present the one or more personalized medicines for heart failure and heart disease on a user interface configured with the computing device.

17. The system for creating one or more personalized medicines for treating heart failure and heart disease of claim 16 wherein the processor is configured to capture the user's PHR data through a PHR module.

18. The system for creating one or more personalized medicines for treating heart failure and heart disease of claim 16 wherein the processor is configured to estimate toxicity of the one or more personalized medicines through a toxicity estimation module.

19. The system for creating one or more personalized medicines for treating heart failure and heart disease of claim 16 wherein the processor is configured to capture the user's bio-sample data through a bio-sample module.

20. The system for creating one or more personalized medicines for treating heart failure and heart disease of claim 16 wherein the system registers the user via a registration module by receiving, processing and storing one or more credentials from the user so that access can be provided to the user.

21. The system for creating one or more personalized medicines for treating heart failure and heart disease of claim 16, wherein the processor is configured to estimate toxicity of the one or more personalized medicines through a toxicity estimation module.

22. The system for creating one or more personalized medicines for treating heart failure and heart disease of claim 16, wherein the processor is configured to process data using a generative adversarial network through a machine learning module.

23. The system for creating one or more personalized medicines for treating heart failure and heart disease of claim 16, wherein the memory is configured to capture a user's medicine adherence data through a medicine plan adherence module.

24. The system for creating one or more personalized medicines for treating heart failure and heart disease of claim 16, wherein the system facilitates communications between the user and an expert through a communication module.

25. The system for creating one or more personalized medicines for treating heart failure and heart disease of claim 16, wherein the system further provides non-pharmacologic treatment recommendations through a non-pharmacologic treatment module.

25. The system for creating one or more personalized medicines for treating heart failure and heart disease of claim 16, wherein the computing device further comprises a micro-electro-mechanical sensor to compute a change in non-invasive intracardiac pressure measurement as a difference between two or more intracardiac pressure measurements.

26. The system for creating one or more personalized medicines for treating heart failure and heart disease of claim 16, wherein the patient data comprises one or more of the following: non-invasive intracardiac pressure measurements, demographic data, bio-sample data, and PHR data.

27. A system for creating one or more personalized medicines for treating heart failure and heart disease, the system comprising:
a processor;
a memory communicatively coupled to the processor, wherein the memory stores instructions executed by the processor, wherein the system is configured to:
capture non-invasive intracardiac pressure signals of the user through a micro-electro-mechanical sensor configured to a computing device;
capture a user's bio-sample data through a bio-sample module;
capture the user's demographic data through a demography module;
capture the user's PHR data through a PHR module;
process the user's patient data in order to create one or more personalized medicines for heart failure and heart disease through a machine learning module, wherein the user's patient data comprises one or more of the following: non-invasive intracardiac pressure signals, bio-sample data, demographic data, and PHR data; and
present the one or more personalized medicines for heart failure and heart disease on the computing device's user interface.

28. The system for creating one or more personalized medicines for treating heart failure and heart disease of claim 27, wherein the bio-sample module is configured so that a user's saliva from salivary glands can be collected from the user and used as a bio-sample.

29. The system for creating one or more personalized medicines for treating heart failure and heart disease of claim 27, wherein the user's bio-sample is placed on a reactant paper having one or more reactant properties pertaining to chemical information of the user's body so that the chemical information can be used as bio-sample data.

30. The system for creating one or more personalized medicines for treating heart failure and heart disease of claim 27, wherein the bio-sample module further captures an image of the reactant paper to obtain the bio-sample data.
